(19) United States
(12) Patent Application Publication
(10) Pub. No.: US 2011/0224405 A1
(43) Pub. Date: Sep. 15, 2011

(54) DEGRADABLE SUPPORTS FOR TIDE SYNTHESIS

(75) Inventors: Andrew Guy Livingston, Knebworth (GB); Ludmila Georgieva Peeva, London (GB); Sheung So, London (GB)

(73) Assignee: IMPERIAL INNOVATIONS LIMITED, London (GB)

(21) Appl. No.: 13/129,396

(22) PCT Filed: Nov. 12, 2009
(86) PCT No.: PCT/GB09/51525

§ 371 Date: May 13, 2011

(30) Foreign Application Priority Data
Nov. 14, 2008 (GB) 0820865.4

(51) Int. Cl. C07K 9/00; C07K 1/04; C07H 21/00

(52) U.S. Cl. 530/322; 530/334; 536/25.3

(57) ABSTRACT

According to the present invention, there is provided a process for synthesis of a first compound selected from peptides, oligonucleotides, and peptide nucleic acids, which comprises synthesis of the first compound linked to a soluble support, wherein the soluble support is degraded following the synthesis so that it can be separated from the first compound.

### FIELD OF THE INVENTION

The present invention relates to a method for the synthesis of compounds, in particular compounds selected from peptides, oligonucleotides, and peptide nucleic acids.

### BACKGROUND TO THE INVENTION

Peptides, oligonucleotides and peptide nucleic acids, hereafter collectively referred to as tides, are biologically important polymers made up of distinct repeat units. In the case of peptides the repeat units are amino acids or their derivatives, while in the case of oligonucleotides the repeat units are nucleotides or their derivatives. Oligonucleotides can be further divided into RNA oligonucleotides and DNA oligonucleotides, as is well known to those skilled in the art, see for example P. S. Millar, Bioconjugate Chemistry, 1990, Volume 1, pages 187-191. In the case of peptide nucleic acids (PNA) the backbone is composed of repeating N-(2-aminoethyl)-glycine units linked by peptide bonds. The various purine and pyrimidine bases are linked to the backbone by methylene carbonyl bonds. The sequence of the amino acids in a peptide, the sequences of RNA nucleotides in RNA or DNA nucleotides in DNA, or the sequence of purine bases in PNA, determine the function and effects of these tides in biological systems.

Tides are synthesised through coupling together their repeat units to give a specific sequence. The repeat units may be protected at one or more reactive sites using protecting groups, to direct coupling reactions to a specific reactive site on the protected repeat unit. Deprotection reactions may be required after a coupling reaction to remove protecting groups and prepare the tide for a subsequent coupling reaction. Tide synthesis takes place in a sequence of cycles, each cycle comprising a coupling reaction followed by a deprotection reaction. Between reactions, the removal of traces of excess reagents and reaction by-products to very low levels is necessary to prevent erroneous sequences being formed in the sequence of repeating units. When the coupling or deprotection reactions are carried out in liquid phase, referred to as liquid phase synthesis, this purification is often tedious and is achieved by time consuming precipitation, crystallisation, or chromatography operations. At the conclusion of the tide synthesis, the desired product tide may be purified by separation from other tides containing error sequences. The chemistries and methods available for coupling and deprotection of peptides, oligonucleotides and peptide nucleic acids, and purification of these tides, are known to those skilled in the art.

Peptide synthesis was revolutionised in 1963 by the advent of solid phase synthesis (Merrifield R B J Am Chem Soc 8.5, (1963) 2149). In this approach, the first amino acid in a sequence is bound to a resin bead. Subsequent amino acids are coupled to the resin bound peptide, and finally, when the desired peptide has been grown, it is cleaved from the resin. Importantly, at the end of each coupling or deprotection reaction, residual unreacted protected amino acids, excess reagents, and other side products can be removed by washing. This includes washing the resin on a filter or flushing a packed bed of resin with solvent. Solid phase peptide synthesis is now a standard technology for laboratory and commercial syntheses. The synthesis of oligonucleotides has followed a similar technological development to peptides, as described by Sanghvi, Y S, Org Proc Res & Dev 4 (2000) 168-169, and relies on solid phase synthesis in which a first oligonucleotide is linked to a solid phase. Further oligonucleotides are attached via cycles of coupling and deprotection reactions, with purification between the reactions carried out by washing. This includes washing the resin on a filter or flushing a packed bed of resin with solvent.

Liquid phase tide synthesis has also developed. Soluble supports including polystyrene, polyvinyl alcohol, polyethyleneimine, polyethylene glycol, polyacrylic acid, polyvinyl alcohol-poly(1-vinyl-2-pyrrolidinone) co polymers, cellulose, and polyacrylamide, have been described for use in methods for facilitating separation of growing peptides and oligonucleotides from excess reagents and reaction by-products by D J Gravert and K D Janda, Chemical Reviews, 1997 Vol 97 pages 489-509. The use of membranes during liquid phase peptide synthesis to separate growing peptides from excess reagents and reaction by-products was reported in U.S. Pat. No. 3,772,264. Peptides were synthesised with poly(ethylene glycol) (PEG) as a soluble support, and separation of the growing peptide chain from impurities was achieved with aqueous phase ultrafiltration. The separation required evaporation of the organic solvent after each coupling step, neutralisation followed by evaporation after each deprotection, and then for either coupling or deprotection, water uptake before ultrafiltration from an aqueous solution. Water was then removed by evaporation and/or azeotropic distillation before re-dissolving the PEG anchored peptide back into organic solvent for the next coupling or deprotection step.

In U.S. Pat. No. 3,772,264, peptides were synthesised linked to polyethylene glycol as a soluble support, which enlarged the product peptide and facilitated separation by the membrane. At the conclusion of the synthesis, the peptide was separated from the soluble PEG support through cleavage at the linker molecule using aqueous solutions of trifluoroacetic acid (TFA), 70 wt % or 95 wt % TFA, followed by addition of diethyl ether to precipitate the peptide from solution.

Soluble supports have also been used in oligonucleotide synthesis. Bonora et al. (Nucleic Acids Research, Vol 18, No 11, 3155 (1990)) have reported using PEG as a soluble support for growing oligonucleotides through the phosphotriesters approach. Soluble PEG supports were linked to an initial dinucleotide, and sequential addition of further dinucleotides was carried out through coupling and deprotection chemistry performed in dichloromethane as a solvent. In between each of these steps, purification of the soluble support-oligonucleotide complex was achieved by precipitation from the dichloromethane solution through addition of diethyl ether. It is claimed that the PEG soluble support led to improved properties of the solids formed during these precipitation steps, with consequent overall process improvements.

Soluble supports can be linked to tides through chemistries known to those skilled in the art, and including those described in the references above. When employing these chemistries, a linker molecule can be inserted between the soluble support and the tide which is amenable to cleavage under conditions where the protected tide remains stable. The tide may be cleaved from the support, and then the soluble support and the tide are separated. Achieving this separation by precipitation of the tide may be difficult when the soluble support and the tide both precipitate from solution with the same anti-solvent. For example, protected peptides and PEG both precipitate from DMF or NMP reaction solutions when diethyl ether is added.

Further, to prepare the soluble support, one end of the linker molecule may be joined to the soluble support, followed by attachment of the initial tide building block to the other end of the linker molecule. However, during the process of attaching the linker molecule to the soluble support, some fraction of the soluble support may remain unreacted.

Solid phase synthesis is therefore generally preferred because of a number of problems in using liquid phase synthesis. Generally these relate to isolation of the product or the need to ensure that the support itself remains intact. If the integrity of the support cannot be ensured during the synthetic steps then the whole synthesis is put at risk. For this reason, where liquid systems are actually used the support is most often a PEG soluble support. These are known to be robust and inert so they can withstand the synthetic process and cleavage of the tide. In addition, it is known that PEG is biologically well tolerated and the resulting tide may be left bound to the PEG as it is not detrimental in vivo. Indeed, the presence of the PEG support can be used to modify the release and binding properties of the tide in vivo.

The problems at the end of the tide synthesis in a liquid system, when the product tide and the unreacted or cleaved soluble support must be separated, have meant that such methods have not been developed to any great extent. Precipitation is the preferred technique, but if the soluble support and the product tide are both precipitated by the same anti-solvent, they cannot be easily separated and other techniques, for example chromatography, may be required. Consequently many workers prefer simply to avoid this method.

WO2005113573 discloses a means of using a degradable support material for tide synthesis. This work teaches that siliceous organic or inorganic materials can be used as supports for tide synthesis. Through careful selection, these support materials can be degraded by reaction with hydrogen fluoride to volatile silicon-fluorine compounds at the end of the tide synthesis. The silicone-fluorine compounds are evaporated from the reaction solution to provide the tide product. This work reduces this technique to practice for solid phase synthesis, but does not demonstrate the technique for liquid phase synthesis. However, hydrogen fluoride is a harsh reagent that presents a number of practical problems for its use—including the inherent health and safety issues of using the material, material compatibility with process equipment, etc.—as well as technical problems for tide chemistry, i.e. hydrogen fluoride is a powerful agent for deprotecting amino acids which may lead to unwanted deprotection during the tide synthesis and the generation of the incorrect tide sequence. The process described in this work using siliceous supports that generate volatile compounds upon degradation with hydrogen fluoride severely limits the range of supports that can be used and potentially limits the chemistries and products that can be made using this process.

The present invention addresses the limitations of the prior art through combining the use of degradable soluble support materials for synthesising tides with membrane filtration. By using membrane filtration, it is possible to select from a wide range of degradable support materials appropriate for the particular tide chemistry and product, which can be degraded under conditions that do not affect the protected groups on the growing tide and tide product. Furthermore, the act of degrading the support at the end of the synthesis enhances the membrane filtration by reducing the size of the species that must pass through the membrane relative to the tide product that must be retained by the membrane. In particular, this is of significant benefit if the membrane selectivity for the intact support material is similar to the tide product—i.e. the selectivity of the membrane for the tide product can be greatly enhanced by degrading the support material and making it smaller. The present invention is able to use a variety of mild reagents to effect degradation of the support. In particular, it is not necessary to use hydrogen fluoride in this procedure or similar reagents.

The present invention aims to provide an improved process for synthesising tides in the liquid phase using soluble supports. It is a further aim to provide a process in which the resulting products can be easily separated from any unreacted material, materials present as a result of the use of the soluble support, etc after synthesis and cleavage of the tide. It is another aim to provide a process that does not require the use of chromatography for isolation of the final tide product. It is thus an aim to provide a process in which the final separation can be achieved by membrane filtration.

The present invention satisfies some or all of these aims.

According to the present invention, there is provided a process for the preparation of a first compound selected from the group comprising: peptides, oligonucleotides and peptide nucleic acids, the process comprising the steps:
(a) providing a soluble support and linking to it a precursor component of the first compound;
(b) synthesising the first compound bound to the soluble support starting from the precursor component;
(c) degrading the soluble support after formation of the first compound to form one or more soluble support degradation products; and
(d) isolating the first compound from at least one of the degradation products of the soluble support using a membrane that is stable in the process solution and which provides a rejection for the first compound that is greater than the rejection of at least one of the degradation products of the soluble support.

The tide, i.e the first compound, may be cleaved from the soluble support either before, after or simultaneously with degradation of the soluble support. Usually, degradation occurs after cleavage of the tide from the support.

The process may include one or more additional optional steps between any of the above steps and/or after conclusion of the process.

We have found that it is possible to degrade soluble supports on completion of the synthesis and that at least one of the degradation products can be separated from the tide. Thus, by incorporating the degradation step, the process of the invention enables easy synthesis and separation of tides in the liquid phase.

The process of the invention enables the use of a support for the synthesis and build up of a tide yet also allows efficient isolation of the tide at the end of the process without the need for chromatography. The process of the present invention thus uses a support which is inert during the synthetic build up of the tide and yet which is subject to chemical attack and degradation in order to allow separation of the peptide in the desired manner without the use of chromatography.

In each case, in the various prior methods for tide synthesis, the synthetic procedures for building up the first compound commence with linking of a precursor component of the first compound to the soluble support via a linking group. The identity of the precursor component depends on the identity of the eventual target tide molecule. Suitable precursor components, i.e. tide building blocks are well known in the art. Subsequent reaction of the linked precursor component allows synthesis of the tide in the manner established in the prior art. The present invention relies on the same initial linking of a precursor component of the target tide molecule and subsequent reaction to form a tide. However, to date it has not been possible in a liquid phase system to conduct simultaneously reactions to form a tide in the presence of support which is then later deliberately degraded. This degradation of the support is achieved without destroying the resulting tide.

### BRIEF DESCRIPTION OF THE DRAWINGS

FIG. 1 shows a general scheme for the production of peptides using membrane enhanced peptide synthesis in conjunction with a degradable soluble support;

FIG. 2 shows a synthetic route for synthesis of polylactide;

FIG. 3 shows the results from hydrolysis of polylactide;

FIG. 4 shows a method for coupling Fmoc protected amino acids to polylactide;

FIG. 5 shows NMR data demonstrating that Fmoc-Ala is linked to a polylactide;

FIG. 6 shows a method for deprotecting Fmoc-Ala-PL-Ala-Fmoc prior to attachment of HMPA to form a Soluble Support-Linker complex.

FIG. 7 shows the synthesis of (HMPA-Ala)₂ poly(lactide) from (Ala)₂ polylactide.

FIG. 8 shows the apparatus used for membrane enhanced tide synthesis.

FIG. 9 shows the synthesis of (HMPA-Ala)₂-Polycaprolactone diol.

### DESCRIPTION OF VARIOUS EMBODIMENTS

In an embodiment, the soluble support is degraded at the completion of the synthesis of the first compound by cleaving it from the first compound and causing it to undergo reaction. In a further embodiment, this is a chemical reaction. In a further embodiment, the rate of the degradation reaction is enhanced by a chemical or biological catalyst (e.g. an organometallic species or enzymes). Reactions which may be used to degrade the soluble support include hydrolysis, oxidation, reduction, and other reactions known to degrade polymeric materials. It is important for the claimed process that the degradation reaction does not adversely affect the first compound.

In a preferred embodiment, the first compound is separated from at least one of the degradation products of the soluble support by membrane filtration in which the first compound is retained on a membrane through which at least one of the degradation products of the soluble support permeate, employing a membrane which provides a rejection for the first compound which is greater than the rejection for at least one of the degradation products.

Chromatography, precipitation, liquid-liquid extraction and adsorption can also be used in conjunction with membrane filtration as a separation means, if desired, in the conduct of the process of the present invention.

In one embodiment, the first compound is synthesised by linking an initial tide building block to a soluble support, and then subsequently carrying out one or more coupling or deprotection reactions in a liquid phase, wherein separation of the tide-soluble support complex from at least one of the reaction by-products and excess reagents after the one or more coupling or deprotection reactions in the liquid phase is carried out by precipitation of the tide-soluble support complex from the post reaction mixture.

In yet another preferred embodiment, precipitation of the tide-soluble support is induced by the addition of an anti-solvent for the tide-soluble support complex.

In yet a further embodiment, the tide-soluble support complex is separated from at least one of the reaction by-products and excess reagents by adding a solvent to create a two liquid phase system in which the tide-soluble support complex preferentially partitions into one liquid phase while at least one of the reaction by-products and excess reagents preferentially partition into the other liquid phase.

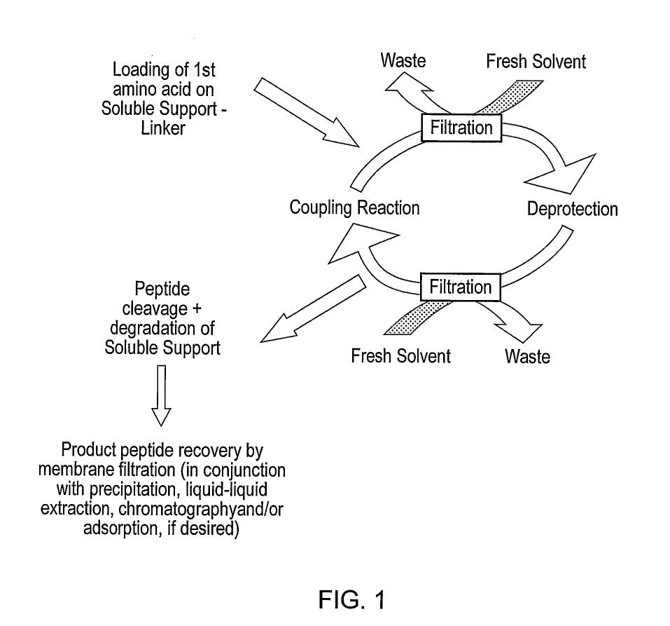

In one embodiment, the first compound is synthesised by linking an initial tide building block to a soluble support, and then subsequently carrying out one or more sequential coupling and deprotection reactions in a liquid phase, wherein separation of the tide-soluble support complex from at least one of the reaction by-products and excess reagents in between at least one combination of sequential coupling and deprotection reactions in the liquid phase is carried out by diafiltration of the post-reaction mixture using an organic solvent, employing a membrane that is stable in the organic solvent and which provides a rejection for the tide-soluble support complex which is greater than the rejection for at least one of the reaction by-products or excess reagents. FIG. 1 shows schematically how the invention may be practised using this embodiment.

In a further embodiment, the organic solvent used for diafiltration is the same as at least one organic solvent present in the liquid phase during the liquid phase synthesis reactions.

In a further embodiment, the organic solvent used for diafiltration is different from at least one organic solvent present in the liquid phase during the liquid phase synthesis reactions.

Suitable soluble supports for use in the present invention include polymers, dendrimers, dendrons, hyperbranched polymers or inorganic or organic nanoparticles. Suitable polymers include materials which are degraded under conditions that are used by those skilled in the art to cleave the first compound from solid or soluble supports, but which are not degraded under the conditions used for coupling and deprotection reactions. Examples include polylactide, polylactide-co-polyglycolide, polycaprolactone diol, polyester, polystyrene, polyvinyl alcohol, polyethyleneimine, polyacrylic acid, polyvinyl alcohol-poly(1-vinyl-2-pyrrolidinone) co polymers, cellulose, polyacrylamide polyamide, polyimide, polyaniline, polymers of terephthalic acid, polycarbonates, polyalkylene glycols including polyethylene glycol, polyethylene glycol esterified with citric acid, copolymers of polyethyleneglycol and succinic acid, of vinylpyrrolidone and acrylic acid or b-hydroxy-ethylacrylate, or of acrylamide and vinylactetate. Polylactide is a particularly suitable support material. Suitable dendrimers for use in the present invention include: poly(amidoamine), also known as PAMAM dendrimers; phosphorous dendrimers; polylysine dendrimers, and; polypropylenimine (PPI) dendrimers which can have surface functional groups including —OH, —NH₂, -PEG, and COOH groups. Nanoparticles may be obtained from commercial sources or synthesised in-situ to provide controlled dimensions, and suitable nanoparticles may be from SiO₂, TiO₂, or other organic or inorganic materials.

U.S. Pat. No. 3,772,264 and UK Patent Application 0814519.5 (filing date 8 Aug. 2008) report suitable chemistries for linking amino acids and peptides to soluble supports. Bonora et al Bioconjugate Chem., (1997) Volume 8 (6), pages 793-797, and Bonora et al (Nucleic Acids Research, Vol 18, No 11, 3155 (1990)) describe chemistries for linking nucleotides and oligonucleotides to soluble supports. Christensen et al. J Pept. Sci. (1995) May-June, 1 (3), pages 175-83 describes suitable techniques for linking peptide nucleic acids to soluble supports. These aforementioned references also describe suitable conditions under which cleavage of the first compound from the soluble support can be achieved.

Suitable chemistries for coupling and deprotection reactions of peptides are well known to those skilled in the art, for example see Amino Acid and Peptide Synthesis, 2^{nd} Edn, J Jones, Oxford University Press 2002, or Schroder-Lubbke, The Peptides, New York 1967. Suitable chemistries for coupling and deprotection reactions on oligonucleotides are well known to those skilled in the art, for example see P. S. Millar, Bioconjugate Chemistry, (1990), Volume 1, pages 187-191 and C. B. Reese Org. Biomol. Chem. (2005), Volume 3, pages 3851-3868. Suitable chemistries for coupling and deprotection reactions of peptide nucleic acids are known to those skilled in the art, for example see B. Hyrup and P. E. Nielsen Bioorganic & Medicinal Chemistry (1996), Volume 4, Issue 1, Pages 5-23. For brevity, the contents of these disclosures as they relate to the present invention are not reproduced here. However, it is specifically intended that the contents of the above references form part of the disclosure of the present invention to the extent that they disclose conditions for linking supports to target materials, and conditions for coupling, deprotection and cleavage. The features of these processes thus can form part of the synthetic process of the present invention.

Suitable membranes for use in the invention include polymeric and ceramic membranes, and mixed polymeric/inorganic membranes. Membrane rejection Rᵢ is a common term known by those skilled in the art and is defined as:

$${R}_{i} = ( 1 - \frac{{C}_{Pi}}{{C}_{Ri}} ) \times 100 \% \qquad ( 1 )$$

where C_{P,i}=concentration of species i in the permeate, permeate being the liquid which has passed through the membrane, and C_{R,i}=concentration of species i in the retentate, retentate being the liquid which has not passed through the membrane.

The membrane of the present invention may be formed from any polymeric or ceramic material which provides a separating layer capable of preferentially separating the tide from at least one reaction by-product or reagent. Preferably the membrane is formed from or comprises a material selected from polymeric materials suitable for fabricating microfiltration, ultrafiltration, nanofiltration or reverse osmosis membranes, including polyethylene, polypropylene, polytetrafluoroethylene (PTFE), polyvinylidene difluoride (PVDF), polysulfone, polyethersulfone, polyacrylonitrile, polyamide, polyimide, polyetherimide, cellulose acetate, polyaniline, polypyrrole and mixtures thereof. The membranes can be made by any technique known to the art, including sintering, stretching, track etching, template leaching, interfacial polymerisation or phase inversion. More preferably, membranes may be crosslinked or treated so as to improve their stability in the reaction solvents. PCT/GB2007/050218 describes membranes which are preferred for use in the present invention.

In a preferred aspect the membrane is a composite material comprising a support and a thin selectively permeable layer, and the non-porous, selectively permeable layer thereof is formed from or comprises a material selected from modified polysiloxane based elastomers including polydimethylsiloxane (PDMS) based elastomers, ethylene-propylene diene (EPDM) based elastomers, polynorbornene based elastomers, polyoctenamer based elastomers, polyurethane based elastomers, butadiene and nitrile butadiene rubber based elastomers, natural rubber, butyl rubber based elastomers, polychloroprene (Neoprene) based elastomers, epichlorohydrin elastomers, polyacrylate elastomers, polyethylene, polypropylene, polytetrafluoroethylene (PTFE), polyvinylidene difluoride (PVDF) based elastomers, polyetherblock amides (PEBAX), polyurethane elastomers, crosslinked polyether, polyamide, polyaniline, polypyrrole, and mixtures thereof.

Yet more preferably the membrane is prepared from an inorganic material such as by way of non-limiting example silicon carbide, silicon oxide, zirconium oxide, titanium oxide, or zeolites, using any technique known to those skilled in the art such as sintering, leaching or sol-gel processing. The inorganic membranes provided by Inopor GmbH (Germany) are preferred for use in this invention.

In a further embodiment, the membrane may comprise a polymer membrane with dispersed organic or inorganic matrices in the form of powdered solids present at amounts up to 20 wt % of the polymer membrane. Carbon molecular sieve matrices can be prepared by pyrolysis of any suitable material as described in U.S. Pat. No. 6,585,802. Zeolites as described in U.S. Pat. No. 6,755,900 may also be used as an inorganic matrix. Metal oxides, such as titanium dioxide, zinc oxide and silicon dioxide may be used, for example the materials available from Degussa AG (Germany) under their Aerosol and AdNano trademarks. Mixed metal oxides such as mixtures of cerium, zirconium, and magnesium may be used. Preferred matrices will be particles less than 1.0 micron in diameter, preferably less than 0.1 microns in diameter, and preferably less than 0.01 microns in diameter.

### EXAMPLES

The following abbreviations are used within the Examples:

| | |
|---|---|
| Di-chloromethane | DCM |
| Di methyl amino pyridine | DMAP |
| Diisopropyl Urea | DIU |
| Diisopropylcarbodiimide | DIC |
| Diisopropylethylamine | DIPEA |
| Dimthylformamide | DMF |
| N-α-Fmoc-L-Alanine | Fmoc-Ala |
| N-α-Fmoc-O-t-butyl-L-tyrosine | Fmoc-Tyr(^{t}Bu) |
| 4-Hydroxymetylphenoxyacetic acid | HMPA |
| N-Hydroxybenzotriazole | HOBt |
| Benzotriazole-1-yl-oxy-tris-pyrrolidino-phosphonium | |
| hexafluorophosphate | PyBOP |
| Poly(lactide) | PL |
| Poly (ethylene glycol) | PEG |
| Polycaprolactone Diol | PCD |
| Tri Fluoro Acetic Acid | TFA |

### Example 1

This example describes the synthesis and then degradation of a soluble polylactide (PL) support suitable for use in the present invention.

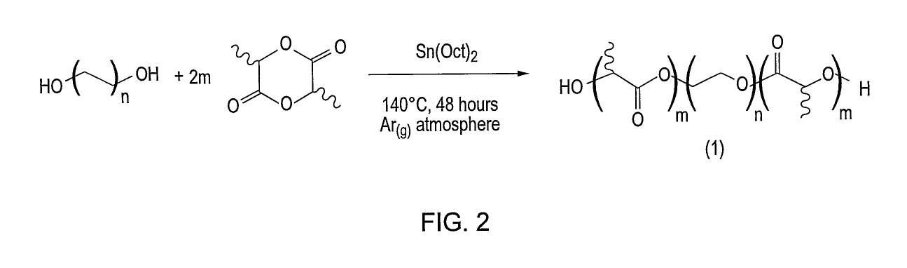

Poly(ethylene) glycol (PEG₂₀₀, molecular weight 200 g.mol⁻¹) was used as the initiator for PL synthesis following the scheme shown in FIG. 2. It was pre-dried in vacuum at 60° C. for 3 hours. Tin(II) 2-ethylhexanoate (Sn(Oct)₂) was employed as catalyst for the synthesis and was used directly from the bottle without drying. 10 g of 3,6-dimethyl-1,4-dioxane-2,5-dione (lactide) was freeze dried before being added into a stainless steel reactor, which contained the pre-dried PEG₂₀₀ (3.6×10⁻³ mol of PEG₂₀₀ per mol of lactide) and Sn(Oct)₂ catalysis (2.9×10⁻⁵ mol of Sn(Oct)₂ per mol of lactide). The final mixture was purged with argon gas before heating to 140° C. for 24-48 hours. Poly(lactide) product (1) was cooled to room temperature and dissolved in chloroform, followed by precipitation and washing with diethyl ether. The polymer was then dried in vacuum for 24 hours. The weight average molecular weight (M_{W}) of the polymer was determined using gel permeation chromatography (GPC) to be 13,500 g.mol⁻¹. The weight average molecular weight M_{W} determined by nuclear magnetic resonance (NMR) was 12,000 g.mol⁻¹.

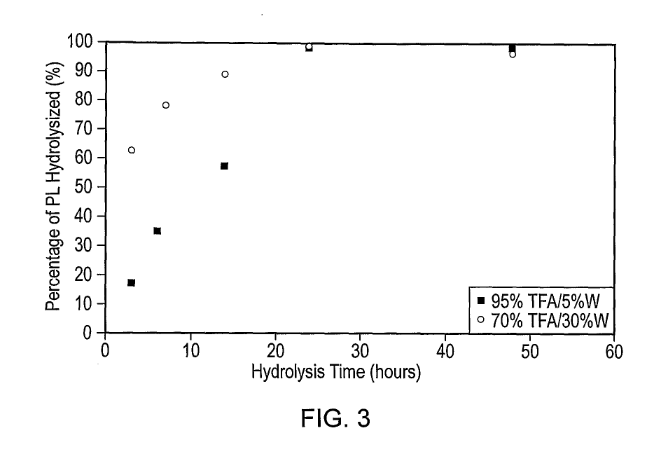

Hydrolysis of the polylactide was performed in aqueous solutions of TFA, 70% TFA/30% H₂O and 95% TFA/5% H₂O. These are the same as conditions commonly used for the cleavage of peptides from soluble and solid phase supports [W. Chan, P. White, Fmoc Solid Phase Peptide Synthesis: A Practical Approach, Oxford University Press (2000); Fischer P, Zheleva D, Liquid-phase peptide synthesis on Polyethylene Glycol (PEG) supports using strategies based on the 9-fluorenylmethoxycarbonyl amino protecting group: Application of PEGylated peptides in biochemical assays. J. Peptide Sci., Vol. 8, (2002), 529-542]. Solid PL was dissolved into the hydrolysis solution. Samples were taken at regular intervals and drowned out with diethyl ether. Un-hydrolysed PL precipitated out upon addition of ether and was then dried under vacuum, while completely hydrolysed PL will become lactic acid which is fully soluble in ether and so did not precipitate out. The results of the hydrolysis experiment are shown in FIG. 3. All PL was fully hydrolysed within 24 hours in the 95% TFA hydrolysis solution. PL hydrolysis was also rapid in 70% TFA/30% H₂O. This data shows that after hydrolysis, it is possible to separate the residues of PL from a peptide through precipitation. The PL residues (lactic acid) are ether soluble, whereas peptides are not and precipitate as a solids.

### Example 2

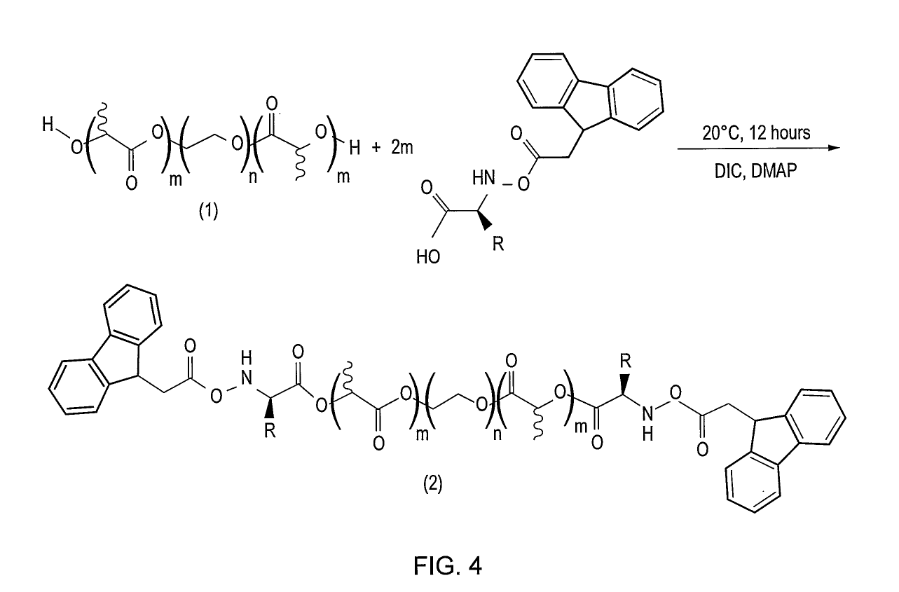

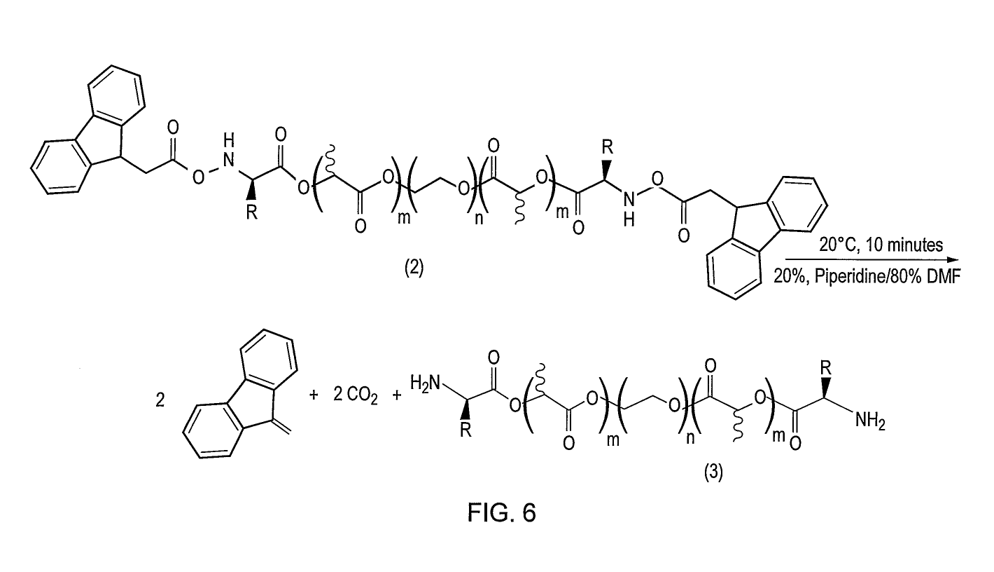

This example describes the attachment of an amino acid, which acts as a linker, to polylactide (PL), following the reaction scheme outlined in FIGS. 4 and 6.

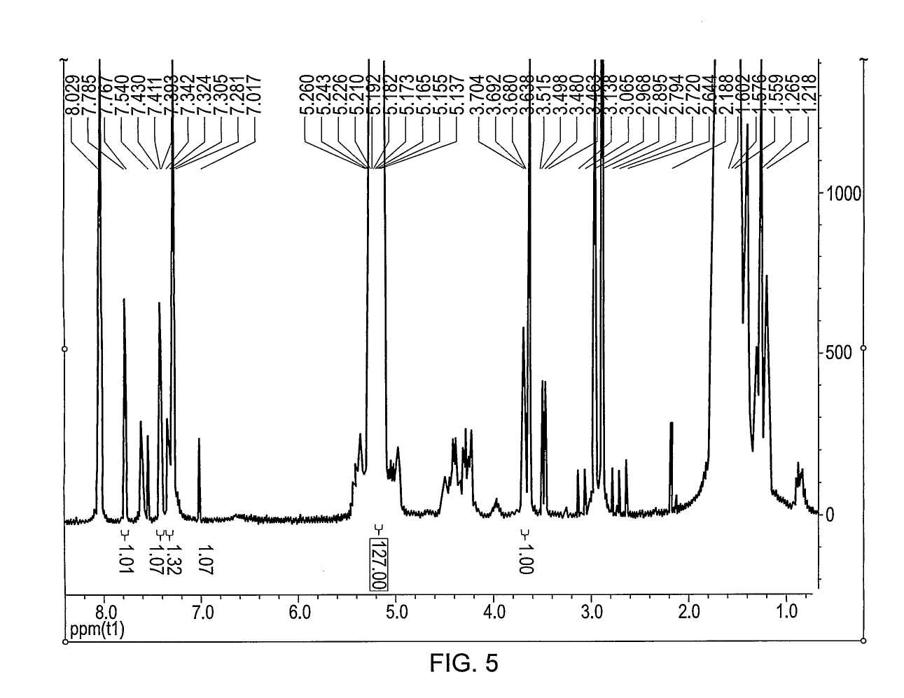

Fmoc-alanine (Fmoc-Ala, 4 mol per mol of PL) and dimethyl-amino-pyridine (DMAP, 0.2 mol per mol of PL) were mixed with the pre-dried PL (1) before dissolving into DMF solvent (5 ml per g PL). Diisopropylcarbodiimide (DIC, 4 mol per mol of PL) was added into the fully dissolved reaction mixture. The coupling reaction as shown in FIG. 4 was performed at 4° C. for 12 hours. Solid diisopropylurea (DIU) was removed by micro-filtration and the coupling reaction was repeated to improve conversion if necessary. Diethyl ether was then added to the product mixture to precipitate (Fmoc-Ala)₂-PL. The conversion of the attachment was determined by NMR analysis and integrating the Fmoc-protecting group at 7.2 (t, 2H), 7.3 (t, 2H), 7.5 (d, 2H) and 7.7 (d, 2H) with the —CH₂— of the PEG₂₀₀ next to the ester bond at 3.6 (t, 4H), as shown in FIG. 5.

The deprotection (removal of Fmoc-groups) from (2) was subsequently undertaken to generate (Ala)₂-PL (3) as shown in FIG. 6. A 20% v/v piperidine/DMF solution was used to remove the Fmoc-protecting groups from (2). Piperidine/DMF solution was added to the pre-dried (Fmoc-Ala)₂-PL solid to form a solution. Deprotection was performed for 20 minutes, followed by precipitation and washing by addition of diethyl ether, recrystallisation by dissolution in DMF/precipitation with ether, and drying in vaccuo. GPC and H¹-NMR were used to verify the disappearance of Fmoc-group at 7.2 (t, 2H), 7.3 (t, 2H), 7.5 (d, 2H) and 7.7 (d, 2H). The Kaiser test was used to confirm the presence of the amino functional groups of the (Ala)₂-PL at the completion of the reaction. The resulting (Ala)₂-PL is suitable for use as in the synthesis of a peptide with Ala as the first amino acid in the sequence.

### Example 3

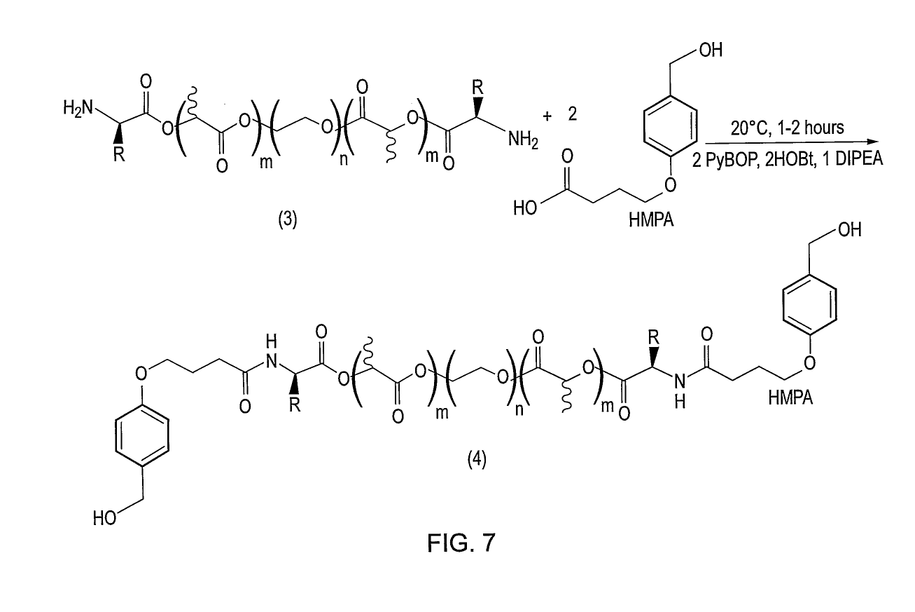

In some cases it may be desirable to place a more labile molecule in the linker to allow more facile cleavage of a product peptide from the soluble support. HMPA may be added to a first amino acid to form an extended linker. Subsequent peptides can then be added to the HMPA. (HMPA-Ala)₂-PL (4) was synthesised as shown in FIG. 7. Pre-dried (Ala)₂-PL (3) prepared as described in Example 2 was dissolved in DCM solvent. 4-Hydroxymethylphenoxyacetic acid (HMPA), PyBOP (both 4 mol per mol (Ala)₂-PL) and DIPEA (2 mol per mol (Ala)₂-PL) were pre-activated in DMF for 15 minutes before being added into the PL solution. The reaction was performed under ambient conditions (20° C., 1 atm. pressure) overnight. The product was precipitated with diethyl ether at 4° C. for 2 hours and separated by centrifugation, followed by ether washes of the recovered product. This crude product was further purified by re-precipitation with DMF/ether followed by chloroform/ether. The (HMPA-Ala)₂-PL product (4) was dried under vacuum and analysed by GPC for the appearance of a UV absorption signal and by H¹-NMR for determining the conversion. The conversion was estimated based on the ratio between peaks at 3.6 (t, 4H) for —CH₂— adjacent to the ester bond and 6.7 (d, 2H), 6.9 (d, 4H) for aromatic system on HMPA linker.

### Example 4

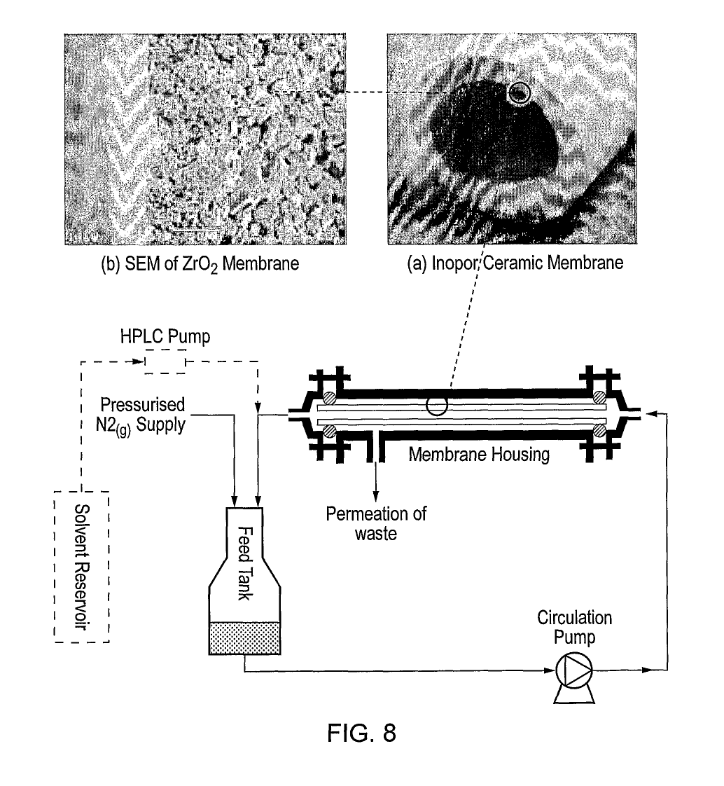

To synthesise a peptide attached to the soluble poly(lactide) support, membrane diafiltration is used for purification of post-coupling and post deprotection mixtures, referred to as Membrane Enhanced Peptide Synthesis (MEPS). The apparatus employed is shown in FIG. 8. Both coupling and deprotection steps are performed in the Reaction Vessel (Feed Tank) at atmospheric pressure. The Circulation Pump recirculates the reaction solution through the membrane cartridge and ensures good liquid mixing throughout. Upon completion of each reaction, the system is pressurised using N₂ to ˜7 barg. The resulting solvent flow permeating through the membrane is balanced by a constant flow of fresh solvent (DMF) supplied to the Reaction Vessel (Feed Tank) from the Solvent Reservoir via an HPLC pump. The same procedure is applied at each reaction/washing cycle. An Inopor zirconium oxide coated membrane with 3 nm pore size and hydrophobic surface modification (Inopor GmbH, Germany) is used to effect purification.

The following steps are performed:

Synthesis of (Fmoc-Tyr-HMPA-Ala)₂-PL. Pre-dried (HMPA-Ala)₂-PL is dissolved in DMF. Fmoc-protected Tyr (Fmoc-Tyr(^{t}Bu), HOBt, DIC (all 4 mol per mol (HMPA-Ala)₂-PL) and DIPEA (1 mol per mol (HMPA-Ala)₂-PL) are pre-activated in DMF for 15 minutes before mixing with (HMPA-Ala)₂-PL solution. The reaction is performed under ambient conditions (20° C., 1 atm. pressure) for 2 hours. Upon reaction completion the excess reagents are removed by constant volume diafiltration (10 volumes of diafiltration solvent per starting solution volume). Permeate samples are collected to monitor losses of PL-peptide and to verify the removal of impurities. At the conclusion of the coupling reaction, small samples of retentate are collected and the PL-peptide precipitated by diethyl ether addition for H¹-NMR analysis to estimate the conversion, and for the Kaiser test to confirm the absence of amino functional groups.

Peptide chain assembly with Fmoc-amino acids. Fmoc-Ala is pre-activated with PyBOP. HOBt (all 2 mol per mol (HMPA-Ala)₂-PL) and DIPEA (1 mol per mol (HMPA-Ala)₂-PL) in DMF solvent for 15 minutes. The pre-activated solution is added into the (Tyr-HMPA-Ala)₂-PL solution. The resulting solution is mixed vigorously for 1 hour followed by a constant volume diafiltration wash (10 volumes of diafiltration solvent per starting solution volume). This procedure is applied for the attachment of further amino acids.

Fmoc-deprotection. 20% piperidine/DMF solution is prepared by adding the required amount of pure piperidine to the known (peptide)₂-PL solution volume. Deprotection is performed for 20 minutes. Purification after each deprotection is performed via diafiltration (12 volumes of diafiltration solvent per starting solution volume).

The coupling and deprotection steps are continued to form the amino acid sequence Fmoc-Tyr-Ala-Tyr-Ala-Tyr-HMPA-Ala-Poly(lactide)-Ala-HMPA-Tyr-Ala-Tyr-Ala-Tyr-Fmoc.

### Side-Chain Deprotection, Peptide Cleavage and PL Support Hydrolysis Reaction.

The solution containing (peptide)₂-PL building block is removed from the MEPS filtration rig, the product is precipitated with diethyl ether and dried in vaccuo. The precipitate is then re-dissolved into the acidolysis solution ((95% TFA, 4% water, 1% protection group scavenger) per mmol of (peptide)₂-PL building block) for 12 hours. This cleaves the peptide at the HMPA linker and hydrolyses the poly(lactide) to lactic acid. Diethyl ether is used to precipitate the purified crude peptide product, with the poly(lactide) degradation products remaining in solution.

### Example 5

In this example polycaprolactone diol (PCD) is prepared as a soluble support and used for peptide synthesis.

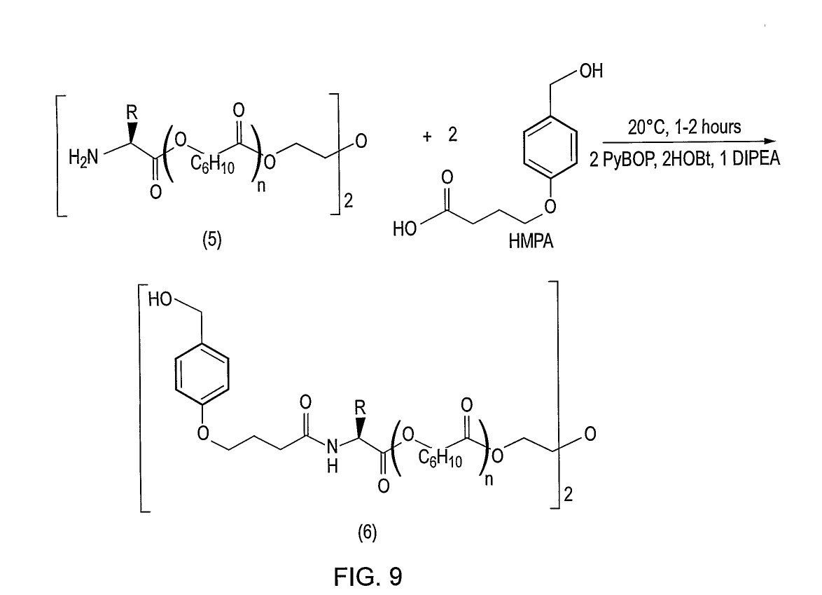

The scheme for synthesis of (HMPA-Ala)₂-PCD (6) is shown in FIG. 9. Pre-dried (Ala)₂-PCD (5) is dissolved in DCM solvent. 4-Hydroxymethylphenoxyacetic acid (HMPA), PyBOP (both 4 mol per mol (Ala)₂-PCD) and DIPEA (2 mol per mol (Ala)₂-PCD) are pre-activated in DMF for 15 minutes before being added into the PCD solution. Reaction is performed under ambient conditions (20° C., 1 atm. pressure) overnight. The product is precipitated with diethyl ether at 4° C. for 2 hours and separated by centrifugation, followed by ether washes. The crude product is purified by recrystallisation with DMF/ether follow by chloroform/ether. (HMPA-Ala)₂-PCD product is then dried under vacuum and analysed by GPC for the appearance of UV absorption signal and by H¹-NMR to determine the conversion.

The (HMPA-Ala)₂-PCD (6) is then used to synthesise peptides following the methods described in Example 4. At the conclusion of the synthesis, the product is precipitated with diethyl ether and dried in vaccuo. The precipitate is then re-dissolved into 20 ml of acidolysis solution (95% TFA, 4% water, 1% protection group scavenger) per mmol of (peptide)₂-PCD building block for 3 hours. Diethyl ether was used to precipitate the peptide product from the liquid phase, with degradation fragments of the PCD remaining in the liquid phase.

1. A process for the preparation of a first compound selected from the group comprising: peptides, oligonucleotides and peptide nucleic acids, the process comprising the steps:
(a) providing a soluble support and linking to it a precursor component of the first compound;
(b) synthesising the first compound bound to the soluble support starting from the precursor component;
(c) degrading the soluble support after formation of the first compound to form one or more soluble support degradation products; and
(d) isolating the first compound from at least one of the degradation products of the soluble support using a membrane that is stable in the process solution and which provides a rejection for the first compound that is greater than the rejection of at least one of the degradation products of the soluble support.

2. A process as in claim 1, in which the soluble support is first cleaved from the first compound, and then degraded.

3. A process as in claim 1, in which the soluble support is degraded by a chemical reaction.

4. A process as in claim 3, where the degradation rate of the soluble support is enhanced by a synthetic or biological catalyst.

5. A process as in claim 1, in which the membrane filtration is performed with microfiltration, ultrafiltration, nanofiltration or reverse osmosis membranes.

6. A process according to claim 1, in which the first compound is synthesised through a series of coupling and deprotection reactions carried out in the liquid phase, and in which precipitation is used for purification of the first compound precursor-soluble support complex after one or more coupling or deprotection reactions.

7. A process according to claim 1, in which the first compound is synthesised through a series of coupling and deprotection reactions carried out in the liquid phase, and in which liquid-liquid extraction is used for purification of the first compound precursor-soluble support complex after one or more coupling or deprotection reactions.

8. A process according to claim 1, in which the first compound is synthesised through a series of coupling and deprotection reactions carried out in the liquid phase, and in which membrane diafiltration is used for purification of the first compound precursor-soluble support complex after one or more coupling or deprotection reactions.

9. A process according to claim 1, in which the soluble support is chosen from polymers, dendrimers, dendrons, inorganic or organic nanoparticles.

10. A process according to claim 9 in which the soluble support is chosen from among polylactide, polylactide-co-polyglycolide, polycaprolactone, polyester, polystyrene, polyvinyl alcohol, polyethyleneimine, polyacrylic acid, polyvinyl alcohol-poly(1-vinyl-2-pyrrolidinone) co polymers, cellulose, polyacrylamide polyamide, polyimide, polyaniline, polymers of terephthalic acid, polycarbonates, poly alkylene glycols including polyethylene glycol, polyethylene glycol esterified with citric acid, copolymers of polyethyleneglycol and succinic acid, of vinylpyrrolidone and acrylic acid or b-hydroxy-ethylacrylate; or of acrylamide and vinylactetate.

11. A process as in claim 1, in which the conditions under which the first compound is cleaved from the soluble support causes the degradation of the soluble support.

12. A process according to claim 1, wherein the membrane is a polymeric membrane.

13. A process according to claim 1, wherein the membrane is a ceramic membrane.

14. A process according to claim 1, wherein the membrane is a mixed matrix organic/inorganic membrane.

15. A process substantially as described in any of the Examples or Figures herein.
